(19) United States
(12) Patent Application Publication
(10) Pub. No.: US 2012/0150275 A1
(43) Pub. Date: Jun. 14, 2012

(54) STENTS AND METHODS OF MAKING STENTS

(75) Inventor: Lori J. SHAW-KLEIN, Rochester, NY (US)

(73) Assignee: MICROPEN TECHNOLOGIES CORPORATION, Honeoye Falls, NY (US)

(21) Appl. No.: 13/315,484

(22) Filed: Dec. 9, 2011

Related U.S. Application Data
(60) Provisional application No. 61/421,951, filed on Dec. 10, 2010.

(51) Int. Cl. A61F 2/82; A61K 9/70

(52) U.S. Cl. 623/1.15; 623/1.38; 427/2.28; 427/2.21

(57) ABSTRACT

The present invention relates to a stent having a longitudinally-extending passage defined by a plurality of seamless strut elements with spacing between them. Each of these strut elements are in the form of lines defining the passage. The strut elements have a thickness in the range of 30 microns to 150 microns and are formed as at least one written layer. Also disclosed are methods of making the stent.

This application claims the benefit of U.S. Provisional Patent Application Ser. No. 61/421,951, filed Dec. 10, 2010, which is hereby incorporated by reference in its entirety.

### FIELD OF THE INVENTION

The present invention relates to stents and methods of making the stents.

### BACKGROUND OF THE INVENTION

Stents may be used to treat stenosis, strictures, or coarctations which are abnormal narrowings in blood vessels, tracts, or other tubular organs or structures in the body. They are most commonly used to treat coronary artery stenosis.

There are various longitudinally-extending passageways in an animal body, which include, for example, blood vessels and other body lumens. These passageways can become occluded or weakened with time or disease. For example, they can be occluded by a tumor, restricted by plaque, or weakened by an aneurysm. When this occurs, the passageway can be reopened or reinforced, or even replaced, with a stent. A stent is an artificial implant that is typically placed in a passageway or lumen in the body.

The stents are delivered inside the body by a catheter. Typically, the catheter supports a reduced-size or compacted form of the stent as it is transported to a desired site in the body (e.g., the site of weakening or occlusion in a body lumen). Upon reaching the desired site, the stent is installed so that it is in contact with the walls of the lumen.

One method of installation involves expanding the stent. The expansion mechanism used to install the stent may include forcing it to expand radially. For example, the expansion can be achieved with a catheter that carries a balloon in conjunction with a balloon-expandable stent reduced in size relative to its final form in the body. The balloon is inflated to deform and/or expand the stent so that it can be placed at a predetermined position in contact with the lumen wall. The balloon can then be deflated and the catheter withdrawn.

When the stent is advanced through the body, its progress can be monitored (e.g., tracked) so that the stent can be delivered properly to a target site. After it is delivered to the target site, the stent can be monitored to determine whether it has been placed correctly and/or is functioning properly. Methods of tracking and monitoring a medical device include X-ray fluoroscopy and magnetic resonance imaging (MRI).

Stent technology has advanced rapidly in response to the pitfalls exposed in each product generation. Bare metal stents, formed of materials such as stainless steel or shape memory alloys, were originally used, but suffer from inflammatory responses leading to renarrowing of the blood vessel. Polymeric drug bearing layers were added to the stent surface in order to slow or prevent such restenosis. However, such drug-eluting stents increase the risk of late thrombosis, thought to be caused by the body's long term reaction to the polymeric material bearing the drug. Other approaches to applying such drugs to the surface of metal stents have been pursued, with varying degrees of success. There is an increasing interest in bioresorbable polymeric stents for coronary, urethral and tracheal applications, where the chance of rejection and thrombosis is thought to be nil. Restenosis may be further minimized for resorbable stents by including a drug bearing coating, included in a bioabsorbable polymer layer or evenly distributed throughout the stent itself.

A number of methods have been developed for the manufacture of stents. Normally an open structure having interconnected struts is preferred from a stent delivery, mechanical, and tissue in growth perspective. Most commonly, metal or polymeric tubes are cast and an optimized perforation structure is formed via laser machining ablation. Such a process is disclosed, for example in U.S. Pat. No. 5,670,161 to Healy et al. Some concerns with this process include sharp edges or burrs, and in the case of polymers, overheating and consequent unintended changes in the microstructure. Other methods for forming the holes have been disclosed, such as water jet cutting (U.S. Pat. No. 5,935,506 to Schmitz et al.) or electrochemical etching (U.S. Pat. No. 5,902,475 to Trozera et al.). All such methods suffer from substantial material waste.

Alternatively, porous stents may be formed from woven, braided or wound metal wires or polymeric filaments. In U.S. Pat. No. 6,245,103 to Stinson, a bioresorbable polymer stent construction is disclosed in which filaments are helically wound and/or braided onto a mandrel, annealed and removed from the mandrel. Such assembly processes may prove tedious and are particularly difficult if bioresorbable filaments of a particular composition are not readily available, or if particular additives must be included (e.g. for radiopacity, drug delivery or mechanical property alteration) but prove difficult to spin into fiber form.

Other inventive methods of producing porous stents have been proposed, including addition of solvent elutable particles to a polymeric matrix, then dissolving them to form an interconnected porous structure (U.S. Pat. No. 4,459,252 to MacGregor), or generation of a membrane by conventional phase separation methods (U.S. Pat. No. 5,527,337 to Stack et al.). Such alternatives offer relatively poor control of perforation size, shape, and uniformity. In addition, there is a limited selection of materials which can be successfully processed in this fashion.

The present invention is directed to overcoming these and other deficiencies in the art.

### SUMMARY OF THE INVENTION

The present invention relates to a stent having a longitudinally-extending passage defined by a plurality of seamless strut elements with spacing between them. Each of these strut elements are in the form of lines defining the passage. The strut elements have a thickness in the range of 30 microns to 150 microns, and are formed as at least one written layer.

The invention also relates to a method of forming a stent. The method involves providing a longitudinally-extending substrate having at least an outer surface. The substrate is formed at least in part from a sacrificial material. The method further involves writing a plurality of spaced strut elements on the outer surface of the substrate. The strut elements collectively form a stent with the sacrificial material being exposed at positions between the spaced strut elements. The writing is carried out with an ink composition. The method also involves removing the sacrificial material from the substrate, leaving the stent having a longitudinally-extending passage defined by the strut elements.

One of the advantages of this invention is the potential to lower manufacturing cost by reducing materials waste and removing manufacturing steps. In addition, the present invention enables greater design flexibility and customization compared with current practices in manufacturing of stents. The methods described in the present invention allow flexibility in the design of strut geometries as well as provide the ability to precisely fine tune strut compositions of stents by altering chemical or physical properties of the inks.

### BRIEF DESCRIPTION OF THE DRAWINGS

FIG. 1 is a perspective view illustrating direct writing of strut elements on the outer surface of a cylindrical substrate.

FIG. 2 is a perspective view, showing the sequence of steps used in a method of removing the substrate after the strut elements have been written on the substrate. After the stent was written on a cylindrical low-surface energy substrate and the ink composition was cured the substrate is removed using physical force. As a result, the stent is left behind.

FIG. 3 is a perspective view, showing the sequence of steps used in a method of recovering a stent written on a cylindrical substrate by either dissolving in a suitable solvent or melting away. As a result the stent is left behind.

FIGS. 4A-C are perspective views, illustrating different strut element patterns that can be written on the substrate.

FIGS. 5A-L illustrate various embodiments of stents of the present invention. FIG. 5A shows a first embodiment of the stent with a longitudinally extending passage shown as dotted lines. FIG. 5B shows cross-section of the first embodiment of the stent taken along line 5B-5B with two layers (FIG. 5C) having different or similar strut compositions. FIGS. 5D, 5E, and 5F show a second embodiment of the stent with three layers. FIGS. 5G, 5H, and 5I show a third embodiment of the stent with an overcoat layer applied on to a single layer. FIGS. 5J, 5K, and 5L show a fourth embodiment of the stent with an overcoat layer applied all around a single layer.

FIG. 6 is a photographic image of a stent produced according to the present invention.

### DETAILED DESCRIPTION OF THE INVENTION

The present invention relates to a stent having a longitudinally-extending passage defined by a plurality of seamless strut elements with spacing between them. Each of these strut elements are in the form of lines defining the passage. The strut elements have a thickness in the range of 30 microns to 150 microns and are formed as at least one written layer.

A seam is defined as a joint consisting of a line, ridge, or groove formed by joining two pieces or two edges of a material along their margins. A seam could be made by fitting, joining, or overlapping together the two pieces or two edges of the material. The stent of the present invention is seamless. The absence of seams in the stents of the present invention provides the stent with enhanced structural integrity which is especially apparent if the stent is made of a material which dissolves or disintegrates under physiological conditions. For example, a dissolvable stent with a seam may lose structural integrity at the seam much faster than other parts of the stent, thereby compromising the function in the stent. Also, a drug eluting stent may release drug at a faster rate at the seam.

The stent of the present invention is designed such that it can mimic the shape and dimensions of various longitudinally-extending passages in the body of a mammal The cross-section of the longitudinally-extending passage could be in any geometric shape such as a circle, a square, a rectangle, or a polygon. One of the advantages of the present invention is the great flexibility in terms of designing the passage and the shape of the stents to permit particular uses. For example, the longitudinally extending passage could be any cross-sectional diameter or shape. In addition, the stent can have a straight tubular passage or one that branches into multiple passages.

The stents of the present invention include implantable or insertable stents (including catheters). They can be a variety of stents having very different uses. Examples of such different stents include coronary vascular stents, aortic stents, cerebral stents, urology stents (e.g., urethral stents and ureteral stents), biliary stents, tracheal stents, gastrointestinal stents, peripheral vascular stents, neurology stents and esophageal stents. The stent is typically an apertured tubular member (e.g., a substantially cylindrical uniform structure or a mesh) that can be assembled about a balloon. The stent usually has an initial small diameter for delivery into the body that can be expanded to a larger diameter by inflating the balloon.

The stents of the present invention can be easily customized to the requirements of patients. For example, it is conceivable that arterial diameter of the patient varies in different regions. Therefore, a suitable stent would have different diameters in different regions and would be shaped such that it fits the vasculature of the patient. Additional physical features such as holes, bends, curves, or flanges can be introduced into the stent so that it is not displaced easily by physiological processes such as vascular pressure or flow.

Depending on the desired application, stents can have a diameter, when expanded for use, of between, for example, 1 mm and 46 mm. A coronary stent can have an expanded diameter of from about 2 mm to about 6 mm. A peripheral stent can have an expanded diameter of from about 4 mm to about 24 mm. A gastrointestinal and/or urology stent can have an expanded diameter of from about 6 mm to about 30 mm. A neurology stent has an expanded diameter of from about 1 mm to about 12 mm. An abdominal aortic aneurysm (AAA) stent and a thoracic aortic aneurysm (TAA) stent have a diameter from about 20 mm to about 46 mm.

In some embodiments, the stent is used to temporarily treat a subject without permanently remaining in the body of the subject. For example, the medical device can be used for a certain period of time (e.g., to support a lumen of a subject) and then can disintegrate after that period of time.

Subjects can be mammalian subjects, such as human subjects (e.g., an adult or a child). Non-limiting examples of tissues and organs for treatment include the heart, coronary or peripheral vascular system, lungs, trachea, esophagus, brain, liver, kidney, bladder, urethra and ureters, eye, intestines, stomach, colon, pancreas, ovary, prostate, gastrointestinal tract, biliary tract, urinary tract, skeletal muscle, smooth muscle, breast, cartilage, and bone.

The strut elements in the stent of the present invention can form an interconnected network. This interconnected network of strut elements usually provides the stent with strength to sustain the physical demands placed on it by physiological processes. The interconnected network of strut elements can be interconnected in many ways as long as they collectively form a longitudinally extending passage. The interconnected network of strut elements can form a mesh, a spiral, or a contiguous cylindrical structure. In one embodiment, the strut elements are in the form of lines extending peripherally around the passage without interruption. These strut elements provide for the structural integrity of the stent. The strut elements can have a thickness in the range of 30 microns to 150 microns with a uniform thickness or varying thickness.

In one embodiment, the stent has at least two written layers. The layers could be deposited on top of each other such that they are joined together at their surface. They can be made of the same material or different materials. Further, the thickness of each layer can be the same or different. Generally, the thickness of the layers are based on the desired physical characteristics of the layer such as physical strength and flexibility. At least one written layer can cover substantially all of the passage or a portion of the passage. It is also possible to write different portions of the passage using different materials.

The written layer is produced from an ink composition. The ink composition usually has a solvent which is removed upon drying or curing. After the solvent is removed, the remaining components of the ink composition form the strut composition. In one embodiment, the stent comprises a plurality of written layers with each different layer having the same strut composition. In another embodiment, the stent comprises a plurality of written layers with at least two layers having different strut compositions.

The ink composition used to write the stent of the present invention comprises at least one polymer. The polymer may also act as a binder for other particulate materials or for other functional additives including drugs, radiopaque materials, or the like. The ink composition comprises a polymer that may be biostable, bioerodable, or bioresorbable so that the stents are, respectively, biostable, bioerodable, or bioresorbable. Such stents could be used in applications like an abdominal aortic aneurysm (AAA) stent, or a bioerodable vessel graft. Bioerodable or bioresorbable materials may be polymeric, ceramic, or metallic. The bioresorbable or bioerodable polymers provide certain advantages relative to biostable polymers such as natural decomposition into non-toxic chemical species over a period of time. Generally, the bioresorbable or bioerodable polymer is selected based on the desired stent resorption or erosion time.

Bioresorbable polymers include, but are not limited to, aliphatic polyesters such as polyglycolide, polylactide, poly(lactide-co-glycolide), polycaprolactone, polybutylene succinate and its copolymers; poly(p-dioxanone) and polytrimethylene carbonate and its copolymers; poly(DTE)carbonate; polyphosphazenes; specific polyester polyurethanes and polyether polyurethanes; polyamides and polyester amides; poly(sebacic anhydride); polyvinyl alcohol; biopolymers such as gelatin, glutens, cellulose, starches, chitin, chitosan, alginates and the like; and bacterial polymers including poly(hydroxybutyrate) and poly(hydroxybutyrate-co-valerate). Functionalized versions of such polymers may be preferred in order to enhance solubility or biodegradation; and copolymers and blends of such materials are common in order to optimize mechanical and chemical properties.

Many metals are bioresorbable under certain conditions, and can be obtained in particulate form appropriate for compounding with a polymeric matrix. Examples of metals which are bioresorbable include magnesium, calcium, zinc, titanium, zirconium, niobium, tantalum, lithium, sodium, potassium, manganese, iron, tungsten, silicon, gold, platinum, iridium, or alloys of these metals. Such metals may prove useful when added to a stent structure by providing mechanical stability, radiopacity, or conductivity in well defined areas or through the entire stent.

All or part of a stent may be formed from polymeric materials which are bioerodable. These materials erode under biological conditions and include polyglycolide, polylactide, poly(lactide-co-glycolide), polycaprolactone, polybutylene succinate, poly(p-dioxanone), polytrimethylene carbonate, polyphosphazenes, specific polyester polyurethanes, polyether polyurethanes, polyamides, polyester amides, poly(sebacic anhydride), polyvinyl alcohol, biopolymers, gelatin, glutens, cellulose, starches, chitin, chitosan, alginates, bacterial polymers, poly(hydroxybutyrate), poly(hydroxybutyrate-co-valerate), functionalized polymers, copolymers, and blends thereof.

Nonpolymeric bioerodable materials include ceramics or glass ceramics. They are most typically used in bone grafting applications, however in light of their erosion in the body may also be contemplated as additives for intentionally degraded vascular prostheses. The most commonly used are generally based on tricalcium phosphate or calcium potassium sodium phosphate. Commercial mixtures of tricalcium phosphate and hydroxyapatite are also commercially available resorbable ceramics (Mastergraft® Resorbable CeramicGranules, available from Medtronic, Inc.). Considering that such materials may be precipitated or ground into fine powders, they can be added to inks intended for forming bioresorbable stents in order to enhance mechanical properties, or added at relatively higher levels in order to introduce porosity or roughness.

All or part of a stent may be formed from polymeric materials which are biostable. These materials do not erode or decompose under biological conditions. Such biostable materials could be epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, poly(p-xylylene), liquid crystal polymer, polymethylmethacrylate, polyhydroxyethylmethacrylate, polyphosphazene, functionalized polymers, copolymers, and blends thereof.

Additives may be present in the ink. Thickeners, viscosifiers, or salts may be added to adjust the rheology and make the stent more easy to manufacture. Surfactants, defoamers, or dispersants may be present in order to facilitate or inhibit spreading on the substrate, improve handling of the ink, improve the quality of the dispersion, or change the coefficient of friction of the dried ink. Particles may be introduced to tune ink rheology; or to introduce roughness or porosity to the stent interior or exterior surface. The ink composition can comprise a metal selected from the group consisting of magnesium, calcium, zinc, titanium, zirconium, niobium, tantalum, lithium, sodium, potassium, manganese, iron, tungsten, silicon, gold, platinum, iridium, and mixtures thereof The ink composition may also comprise a ceramic material selected from the group consisting of tricalcium phosphate, calcium potassium sodium phosphate, tricalcium phosphate, titanium oxide nitrate, hydroxyapatite, and mixtures thereof The ink composition can also comprise one or more surface active agents, rheology modifiers, lubricants, matting agents, spacers, pressure sensors, temperature sensors, chemical sensors, magnetic materials, radiopaque materials, conducting materials, therapeutic agents, or combinations thereof.

To enhance the radiopacity of the stent, radiopaque materials can be added to the stent. Non-limiting examples of such radio opaque materials include magnesium, calcium, zinc, titanium, zirconium, niobium, tantalum, lithium, sodium, potassium, manganese, iron, tungsten, silicon, gold, platinum, iridium, bismuth oxychloride, bismuth bicarbonate, bismuth trioxide, barium sulfate, and mixtures thereof In order to make the ink composition conductive, a conducting material can be added to the ink composition. Non-limiting examples of such conducting material include gold, platinum, silver, nickel, copper, iron, titanium, magnesium, silicon, carbon, graphite, electrically conducting polymers, and mixtures thereof.

In another embodiment, the ink comprises a therapeutic agent. Non-limiting examples of such therapeutic agent include everolimus, sirolimus, zotarolimus, biolimus, pimecrolimus, tacrolimus, trapidil, rapamycin, paclitaxel, antithrombogenic, antiproliferative, antimotic, anti-inflammatory agents, antioxidants, anti-coagulants, anesthetics, antibiotics, and combinations thereof. Therapeutic agents can be used singularly, or in combination. Additional examples of therapeutic agents are described in U.S. Patent Application Publication No. 2005/0216074, which is hereby incorporated by reference in its entirety.

In formulating an ink for stent manufacture, the constituents are generally dissolved or dispersed in a liquid carrier. Any number of organic solvents, water, acids, or bases may be used. As an alternative, the ink can be melt extruded for stent manufacture. Solvents which may be employed in the present invention include: paraffinic hydrocarbons such as cyclohexane; aromatic hydrocarbons such as toluene or xylene; halohydrocarbons such as methylene dichloride; ethers such as anisole or tetrahydrofuran; ketones such as acetone, methyl ethyl ketone or methyl isobutyl ketone; aldehydes; esters such as ethyl carbonate, 4-butyrolactone, 2-ethoxyethy acetate or ethyl cinnamate; nitrogen-containing compounds such as n-methyl-2-pyrrolidone or dimethylformamide; sulfur-containing compounds such as dimethyl sulfoxide; acid halides and anhydrides; alcohols such as ethylene glycol monobutyl ether, a-terpineol, ethanol, or isopropanol; polyhydric alcohols such as glycerol or ethylene glycol; phenols; or water or mixtures thereof. The binder polymer may also be present as an undissolved dispersion, or polymer latex, suspended in water.

Preferred solvents are those which have the lowest toxic potential when left behind in residual quantities, such as acetone, 1-butanol, ethanol, 1-propanol, methyl acetate, anisole, methyl acetate, methyl ethyl ketone, and the like. Combinations of solvents sometimes prove especially useful in obtaining good solubility with minimal risk of toxicity. It is preferable to choose solvents which evaporate at a convenient rate such that the temperature of the stent material and sacrificial substrate can be maintained below their melting points, such that unwanted deformation does not occur during drying or curing.

The present invention can utilize a wide variety of materials, permitting simplified production of multiple layers and flexibility in customization of stent strut and perforation design.

The present invention also relates to a method of forming a stent. The method involves providing a longitudinally-extending substrate having at least an outer surface. The substrate is formed at least in part from a sacrificial material. The method further involves writing a plurality of spaced strut elements on the outer surface of the substrate. The strut elements collectively form a stent with the sacrificial material being exposed at positions between the spaced strut elements. The writing is carried out with an ink composition. The method also involves removing the sacrificial material from the substrate, leaving the stent having a longitudinally-extending passage defined by the strut elements.

The substrate can have a tubular or cylindrical shape and is made of sacrificial material. The sacrificial material can be made of, for example, silicone, polytetrafluoroethylene, graphite, wax, hydroxyethyl cellulose, polyvinyl pyrrolidone, polyvinyl alcohol, polyethylene oxide, poly(ethyl oxazoline), polysaccharides, polyethylene oxide, and proteins.

Writing a plurality of spaced strut elements on the outer surface of the substrate can be carried out by direct writing, using the ink compositions described supra. Direct writing techniques that satisfactorily control and manipulate the substrate may be used for the purposes of the present invention. These include screen printing, jetting, laser ablation, pressure driven syringe delivery, inkjet or aerosol jet droplet based deposition, laser or ion-beam material transfer, tip based deposition techniques such as dip pen lithography, electrospraying, or flow-based microdispensing (e.g., Micropen™ [Micropen Technologies Corp., Honeoye Falls, N.Y.] or NScrypt® technologies). Such techniques are well described in Pique et al., Direct-Write Technologies for Rapid Prototyping Applications: Sensors, Electronics, and Integrated Power Sources, Academic Press (2002), which is hereby incorporated by reference in its entirety. Direct writing techniques used to apply surface layers, such as drug-eluting layers, as described in U.S. Patent Application Publication No. 2006/0155370 to Brister, which is hereby incorporated by reference in its entirety, or biostable layers, as described in U.S. Patent Application Publication No. 2008/0071352 to Weber et al., which is hereby incorporated by reference in its entirety.

Microdispensing (e.g., Micropen™ direct writing) is particularly preferred for marking medical devices due to their ability to accommodate inks having an extremely wide range of rheological properties and very high solids levels, as well as excellent three dimensional substrate manipulation capabilities. As a result, any material which can be successfully dissolved or dispersed in liquid, and forms a continuous layer when dry, can be formed into a stent. Also, the disadvantages of laser machining, including burr formation, sharp edges, inadvertent heating, and material waste are not a concern with Micropen™ direct writing. To form the stent, a Micropen™ direct writing device can be used to apply or deposit the lines of the two or more selected ink compositions in an interconnected or layered structure such that they form struts resulting in a continuous network.

Stent geometries may consist of open or closed cells, both of which have usefulness depending on whether more support or more flexibility is desired. Cells may be large or small, and vary in size across the length of the stent. Strut geometry is understood to affect endothelialization of the stent, with particular edge angles relative to blood flow most desired. Strut thicknesses generally range from about 50 μm to 150 μm and direct writing techniques can accommodate all of these variables and lead to an advantageous design suitable for the ultimate use of the device.

It may be preferable to print on the inside of a hollow tube, which acts as a substrate, rather than on the outside of a cylinder or tube. It is believed that a strut angle of about 30 degrees relative the direction of blood flow may be advantageous from a tissue in growth perspective, and this can be most easily accommodated by printing the stent on the interior diameter of a tube or, alternately, turning the stent inside out after removal from the substrate.

The substrate may be treated before printing in order to optimize wetting or adhesion properties. Common treatments used for such purposes include flame, plasma, or corona discharge treatments. The removal of sacrificial material from the substrate can be carried out by melting, physically removing, disintegrating, or dissolving the sacrificial material.

For removal by melting, the substrate can be chosen such that the melting temperature of the stent is higher than the melting temperature of the substrate. This allows the substrate to melt away upon reaching its melting point, leaving behind an intact and separate stent. For example, wax is useful as a substrate which can be easily melted in order to remove the stent.

Any convenient substrate material may be chosen as long as its melting point is sufficiently below the softening temperature of the stent polymer. Such polymer will withstand the environment chosen for curing or drying the stent ink. For instance, water-soluble polymers such as polyvinyl alcohol, polyvinylpyrrolidone, polyethylene oxide, polyethyloxazoline, hydroxyethyl cellulose,or carboxymethyl cellulose, may be applied by dipping or coating to the surface of any type of substrate.

The stent can also be physically removed from the substrate by using force. To improve the removability of the stent from the substrate or the mold, a compatible release agent, such as soap, was, or a surfactant may be coated on the substrate prior to writing the stent on the substrate. A soluble layer may also be coated on the substrate. This soluble layer is insoluble in the solvent of the polymer solution, while being soluble in any other solvent. For example, sugar or glucose solution can be used as such a soluble layer, which is dissolved in water prior to physically removing the stent.

The substrate also can be physically disintegrated using force or pressure such that it is easier to remove the intact stent.

Alternatively, the substrate may be dissolved in a solvent so that the stent is left behind intact. The solvent used to dissolve the substrate must be selected so that it does not dissolve the stent.

The method of the present invention further comprises applying an overcoat layer covering at least a portion of the surface of the stent. Many different kinds of materials can be used to make the overcoat layer. The overcoat layer can be selected from the group consisting of biomaterials, cellular layer, tissue layer, fabric layer, micromesh metal layer, and ink composition layer. The overcoat layer can also have at least one therapeutic agent. The therapeutic agents described supra can be incorporated into the overcoat layer for this purpose.

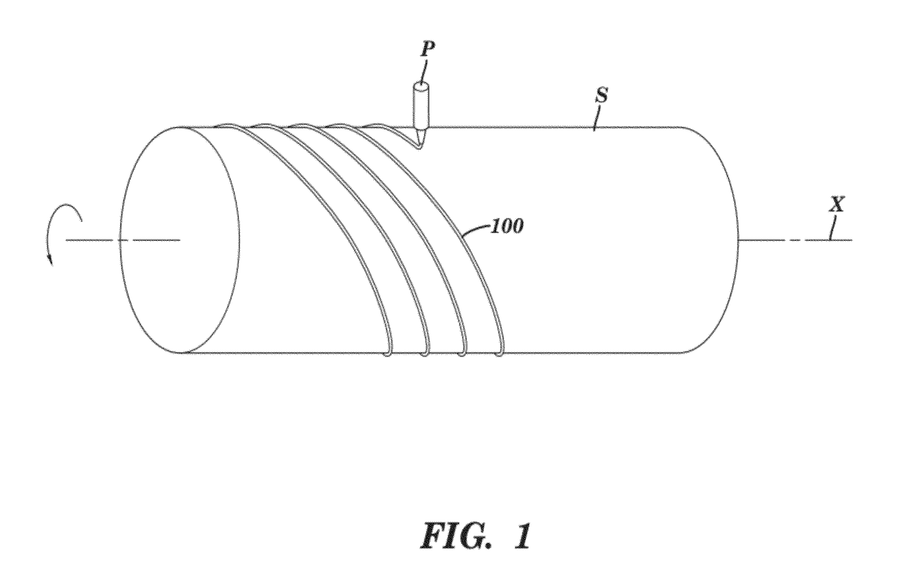

The stent-making inks may be deposited on any number of substrates, as long as the substrate can be subsequently removed without damaging the dried or cured stent. A preferred scenario involves providing a longitudinally-extending substrate as depicted in FIG. 1. Substrates with low surface energy are preferable because they allow for easy removal of the stent. By low surface energy substrate it is meant that the inks can be deposited on the surface of the substrate such that the inks poorly wet the substrate and upon curing or drying there is poor adhesion of the substrate to the stent. The surface energy across an interface or the surface tension at the interface is a measure of the energy required to form a unit area of new surface at the interface. One of the important characteristics of a liquid (or fluid) material is its ability to freely wet the surface of the substrate. At the liquid-solid surface interface, if the molecules of the liquid have a stronger attraction to the molecules of the solid surface than to each other (the adhesive forces are stronger than the cohesive forces), wetting of the surface occurs. Alternately, if the liquid molecules are more strongly attracted to each other than the molecules of the solid surface (the cohesive forces are stronger than the adhesive forces), the liquid beads-up and does not wet the surface of the part.

As shown in FIG. 1, an ink composition is deposited or written on a longitudinally-extending substrate S, for example a solid or tubular cylinder, to form strut element 100. Strut elements 100 can be written in any desired pattern using writing device P. In carrying out this procedure, writing device P can be moved relative to substrate S and/or substrate S can be rotated and translated along axis X, to facilitate the writing of strut elements 100.

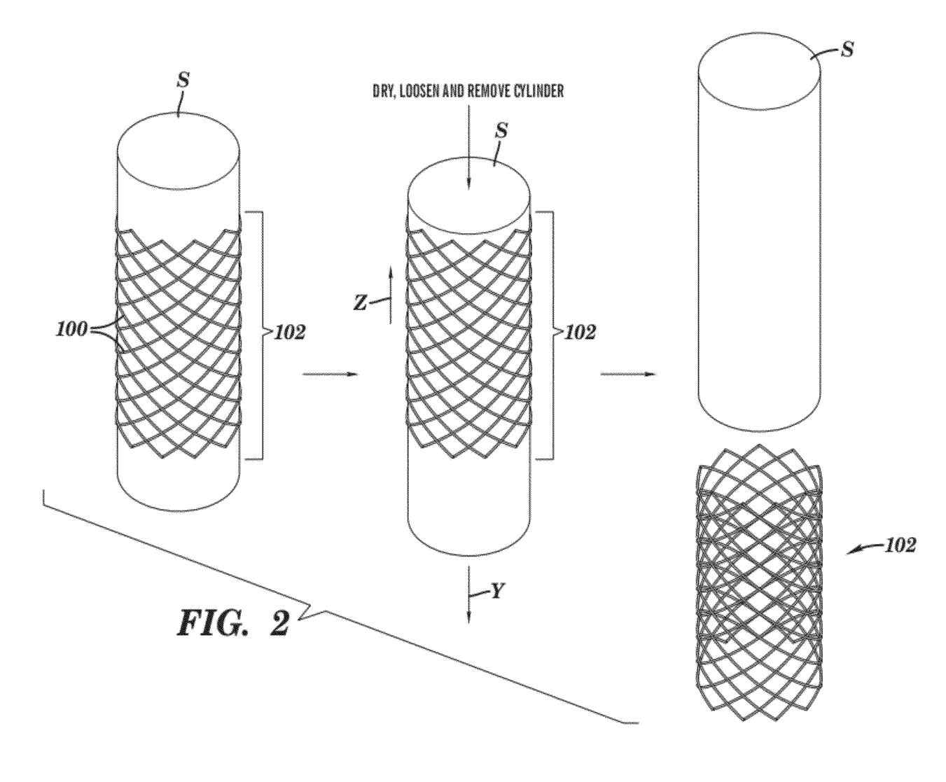

As depicted in FIG. 2, after the ink composition used to write strut elements 100 has been cured or dried, stent 102 is gently loosened from surface of the substrate S, if necessary, and removed from substrate S by sliding the substrate in direction Y or peeling stent 102 by moving in direction Z. Examples of particularly useful materials for a substrate which can be used for removal by sliding or peeling include polytetrafluoroethylene or silicone rubbers. Alternatively, a thin layer of low surface energy material, such as wax, or surfactant, may be applied to the smooth exterior of substrate S to form a release layer. After stent 102 is written and the ink composition is cured or dried, the substrate can be removed as a result of facilitation by the release layer.

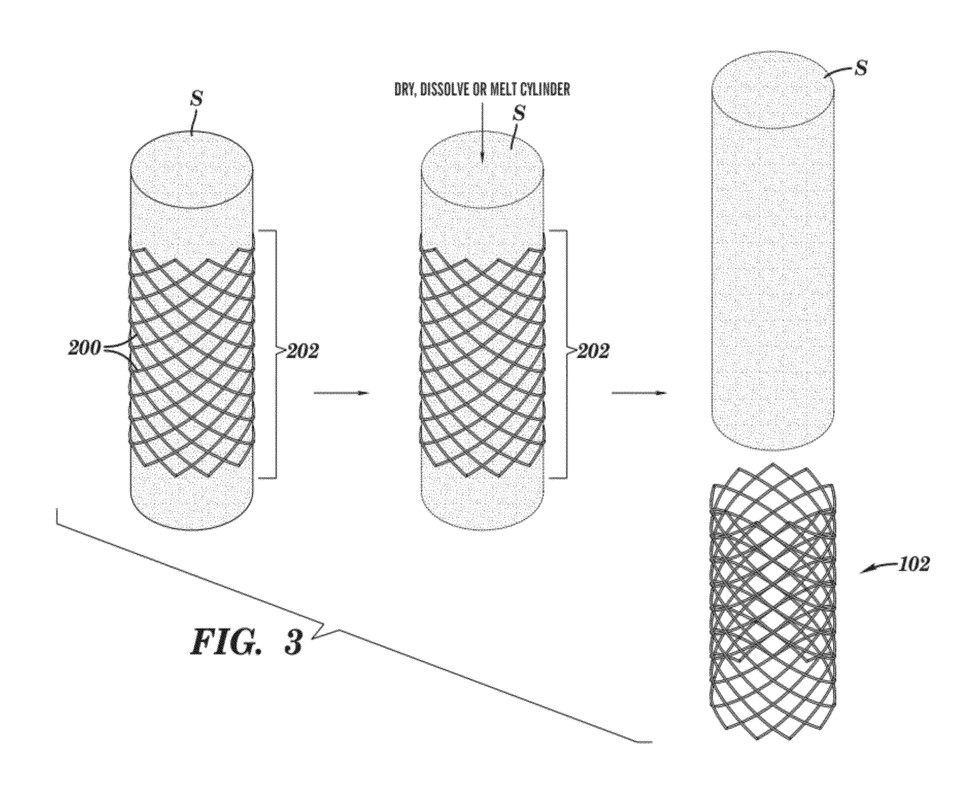

As shown in FIG. 3, substrate S may be designed to dissolve in a solvent or to melt. Entire substrate S can made of soluble material or a material that can be melted without affecting stent 202. Alternatively, substrate S can be coated with a release layer using a soluble material or a material that can be melted in order to remove stent 202. The ink composition is applied on the substrate to write strut elements 200. The ink composition is cured or dried to form strut elements 200. These strut elements 200 form stent 202. Substrate S or a soluble layer is then removed by soaking in a solvent or melting, leaving stent 202.

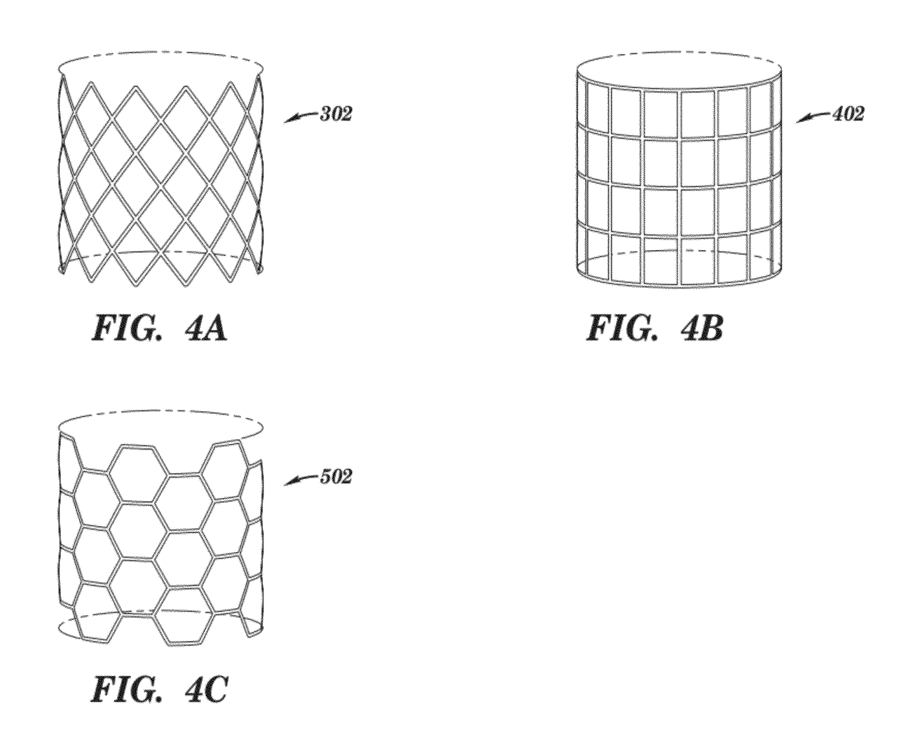

The stents of the present invention can be written in a variety of patterns. FIGS. 4A-C show some examples of stent patterns 302, 402, and 502 which could be employed.

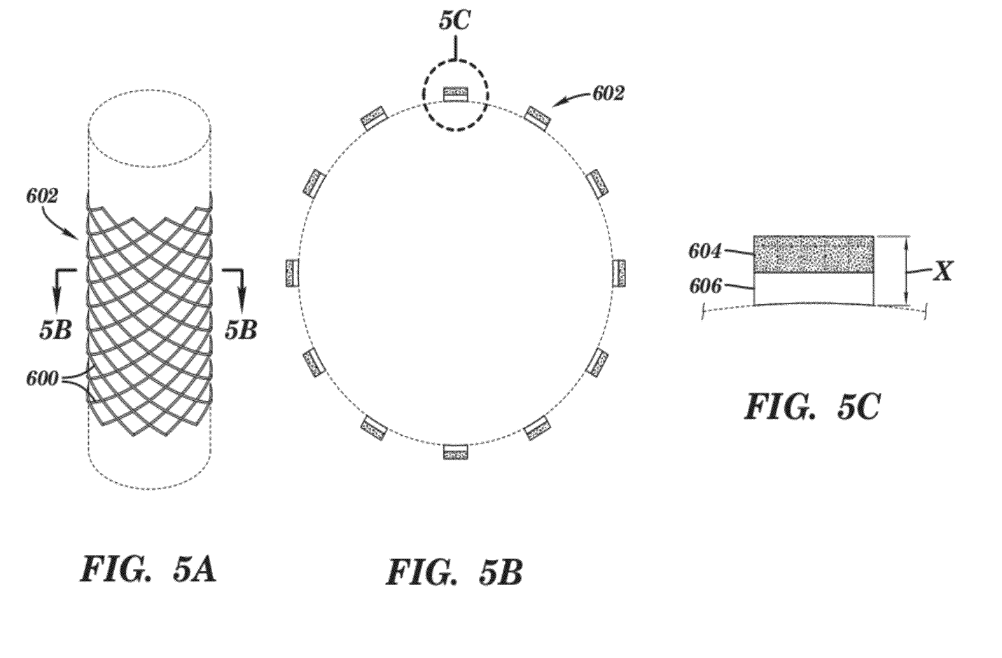

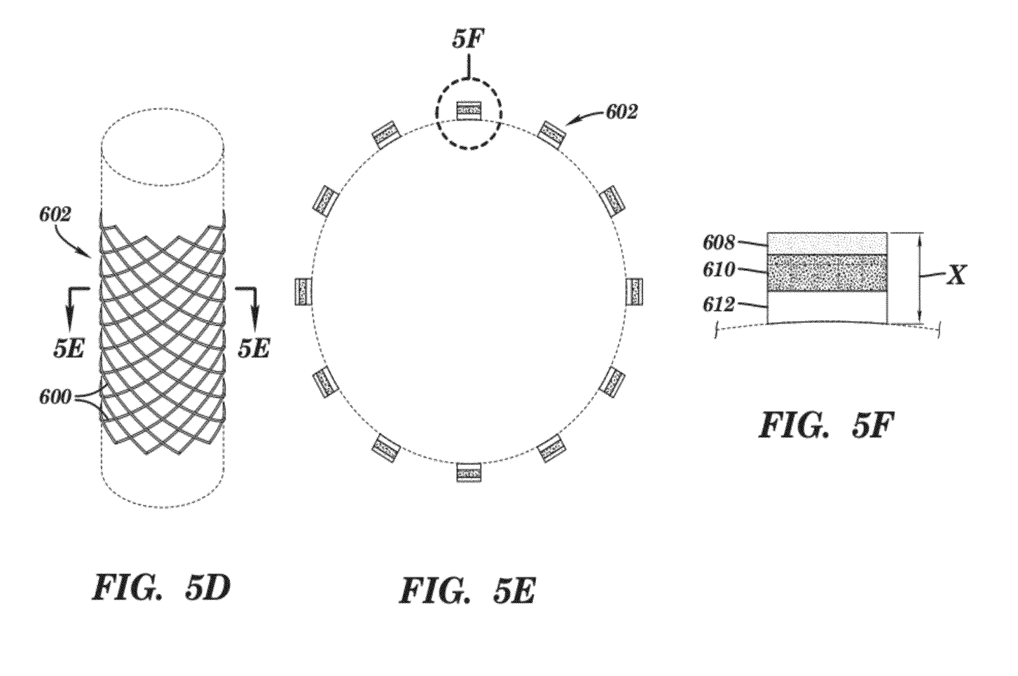

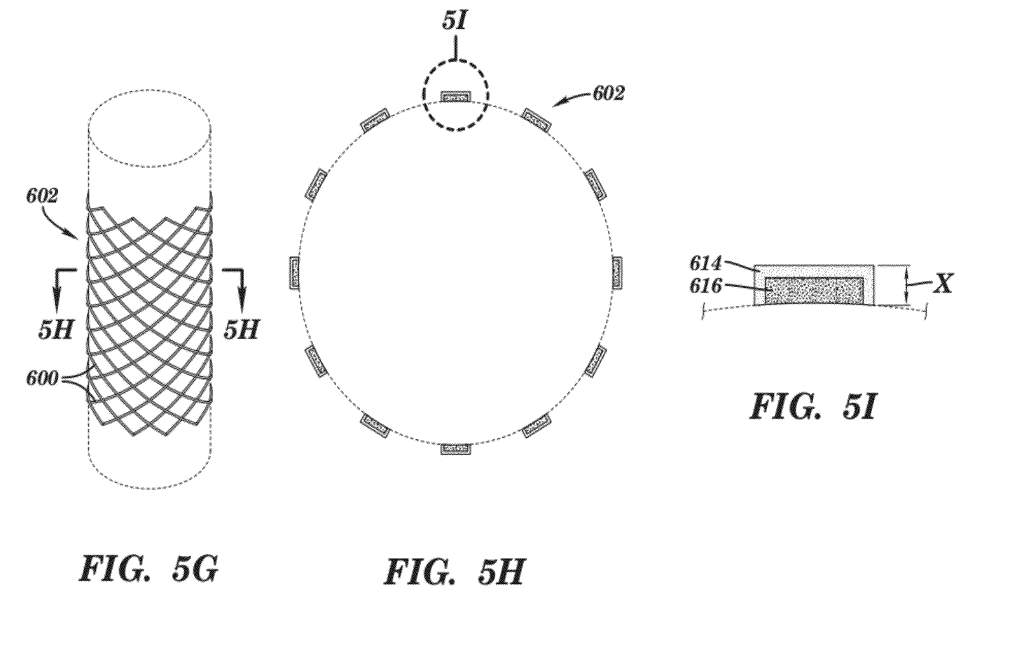

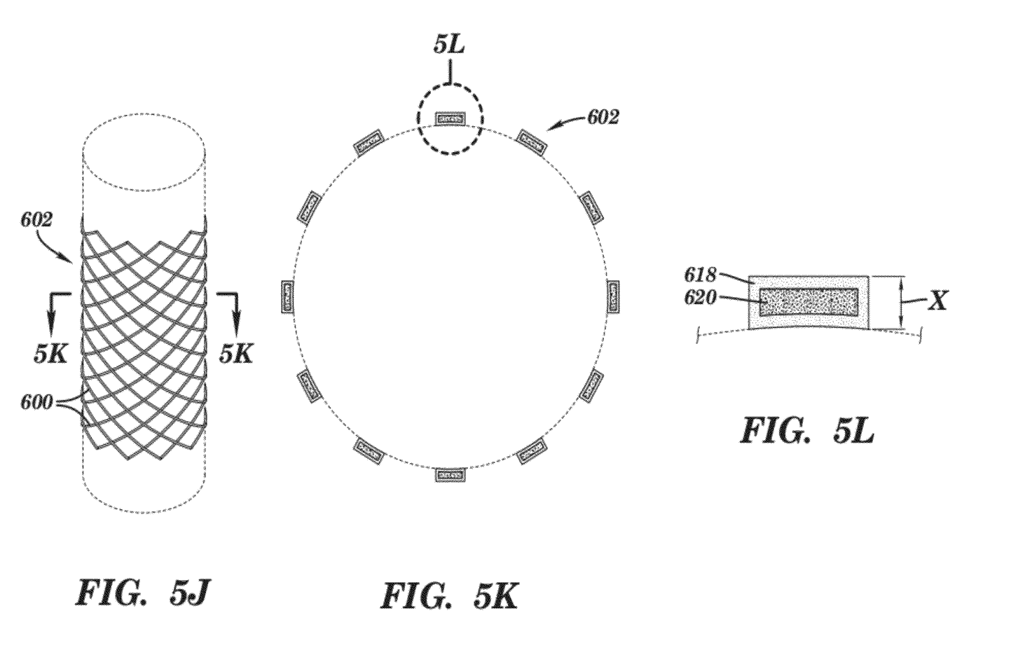

The stents of the present invention have great design flexibility. FIG. 5A illustrates stent 602 with strut elements 600. A cross-sectional view of stent 602, taken along line 5B-5B, is shown in FIG. 5B. Two different layers of strut composition 604 and 606 are shown in FIGS. 5B and 5C (the latter being an enlarged cross section of a strut element). Dimension X is the total thickness of the strut elements. These two layers 604 and 606 are formulated and deposited sequentially, dried, and removed from the substrate in order to form stent 602.

These two layers 604 and 606 are written on top of each other. Each layer could have, for example, a different resorbability rate, different erosion rate, different drug component, coefficient of friction, or a radiopaque component. This can be achieved by appropriately formulating the ink compositions used to write the layers. Further, these layers can be written such that only certain portions of the layers are, for example, radiopaque, bioresorbable, or drug-bearing. For example, half of layer 604 could be written using a composition containing a radiopaque additive making only that portion radiopaque. This is particularly useful in allowing pinpoint accuracy in stent placement. Other additives may be envisioned for which it would be beneficial to segregate on one or more spatial regions of the stent. Alternatively, it may prove beneficial to incorporate different thicknesses in different regions of the stent, enabling, for example, differential resorption times for different stent regions. It may also be desirable to provide different regions of the stent with different mechanical properties. These scenarios are also easily accommodated in the current invention.

Writing portions with different compositions is a cost effective way of using materials. It can be used to make the stent partially resorbable or erodible. Making only small portions of the stent radiopaque could be used to control the visibility of the stent. For example, only erodible or resorbable portions of the stent can be made radiopaque such that the erosion or resorption of the stent may be monitored. In a similar fashion, drug bearing layers can be written such that only a portion of the stent has drug. Such methods can be used for controlling the delivery of drug, for example, the outer layer can have the drug while the inner layers provide the structural strength to the stent. Drug concentration can be controlled by using highest concentration of drug near the free surface. Alternatively, it may be desirable to situate a different drug deeper in the stent structure. With regards to coefficient of friction, by confining the lower surface energy species to a single printing layer, their quantity may be minimized while potentially permitting a less damaging stent insertion.

No limit on the number of layers or composition of the layers is implied. While two layers are shown in this FIG. 5C for illustrative purposes, additional layers can also be contemplated, as shown in FIGS. 5D, 5E (a cross-sectional view of stent 602, taken along line 5E-5E of FIG. 5D), and 5F (shows an enlarged cross section of a strut element). For example, stent 602 has three separate layers 608, 610, and 612. Layers 608, 610, and 612 or portions thereof can be written such that they have different compositions.

FIGS. 5G, 5H (a cross-sectional view of stent 602, taken along line 5H-5H of FIG. 5G), and 51 (the latter being an enlarged cross section of a strut element) show a stent 602 with two layers 614 and 616. Layer 614 forms an overcoat layer and leaves one surface of layer 616 exposed. The overcoat layer is written such that it leaves behind an exposed surface on the single layer (FIG. 5I). This exposed surface could be used for controlled delivery of a drug on the inside of the stent.

Similarly, FIGS. 5J, 5K (a cross-sectional view of stent 602, taken along line 5K-5K of FIG. 5J), and 5L (the latter being an enlarged cross section of a strut element) show a stent 602 with two layers 618 and 620. Layer 618 forms an overcoat layer which completely surrounds layer 620. This can be achieved by, for example, writing all around the strut elements or by immersing the stent in an ink composition that forms the overcoat layer. The overcoat layer could also be applied over multiple layers. The most likely scenario would be to write three layers, the bottom, the middle, and an encapsulating layer of the same composition as the bottom layer.

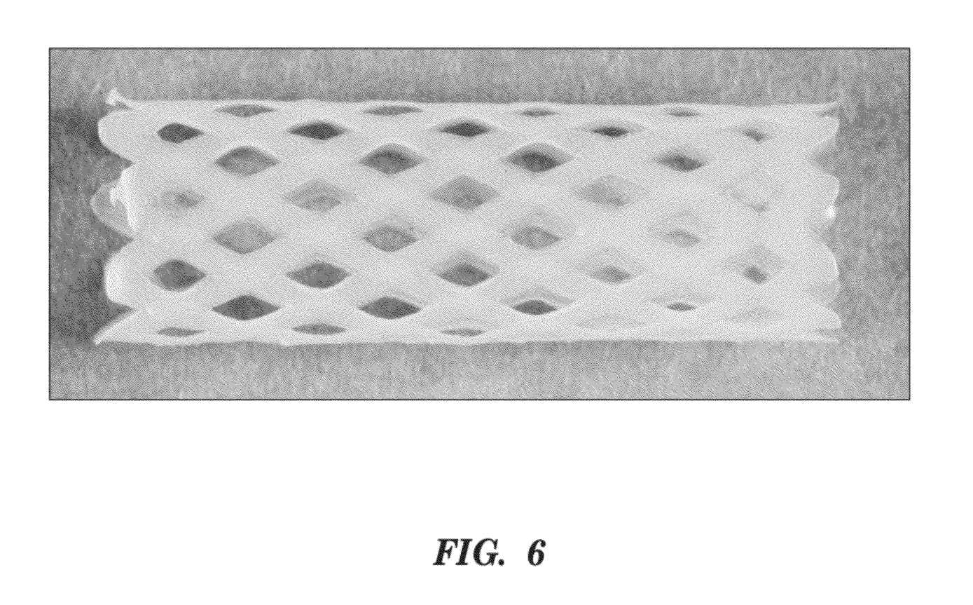

FIG. 6 is a photographic image of a stent produced by a direct write technique on a sacrificial substrate.

### EXAMPLES

### Example 1

### Polycaprolactone Stent

Polycaprolactone (Mn 70,000-90,000; Sigma-Aldrich) was dissolved in tetrahydrofuran (Sigma-Aldrich) at a level of 20% by weight. This stent ink was deposited by a Micropen™ writing device in a continuous open pattern on a polytetrafluoroethylene (PTFE) tube (5 cm outer diameter; Zeus Advanced Biomaterials). A total of four layers were applied, each one positioned directly on top of the previous one. After the first, second, and third layers were applied, the stent was allowed to dry under ambient conditions for approximately 5 minutes before writing the next layer. After the fourth layer was printed, the stent was cured at 55° C. for 10 minutes in a forced air oven. The stent was easily removed in a single piece from the PTFE tube, yielding a device with a thickness of approximately 80 μm and a strut width approximately 0.7 mm. The openings between struts were approximately 1.5 mm in width and 3 mm in length. The entire stent length was approximately 20 mm.

### Example 2

### Radiopaque Polycaprolactone Stent

To the polycaprolactone ink described in Example 1, tungsten powder (99.9%, 1-5 μm, Alfa-Aesar) was added to yield a weight ratio of tungsten:polycaprolactone of 88:12 (volume ratio of 30:70) and a total solids level of 67.6% by weight. A radiopaque stent was produced by writing a single layer of this ink, using a Micropen™ writing device, on a polytetrafluoroethylene (PTFE) tube (3.6 cm outer diameter; Zeus Advanced Biomaterials) and drying at 55° C. for 10 minutes before removing from the PTFE tube. The thickness of the resulting stent was 36 μm, and the length and opening dimensions were identical to that described in Example 1.

### Example 3

### Two-Part Stent that is Radiopaque Only at its Ends

Example 1 was repeated except only two layers of ink were deposited, resulting in a stent approximately 40 mm thick. Subsequently, the tungsten filled ink of Example 2 was deposited only over the struts on either end of the stent. This final product was cured at 55° C. for 10 minutes and removed from the tube.

### Example 4

### Polylactide Coated Stent

Poly(D, L-lactide) (100 DL 7E; Lakeshore Biomaterials) was dissolved in tetrahydrofuran (Sigma-Aldrich) at a level of 33.3% by weight. This stent ink was deposited in a continuous open pattern on a polytetrafluoroethylene (PTFE) tube (5 cm outer diameter; Zeus Advanced Biomaterials). A total of four layers were applied, each one positioned directly on top of the previous one. After the first, second, and third layers were applied, the stent was allowed to dry under ambient conditions for approximately 5 minutes before writing the next layer. After the fourth layer was printed, the stent was cured at 55° C. for 10 minutes in a forced air oven. The stent was peeled in a single piece from the PTFE tube yielding a device with a thickness of approximately 130 μm and a strut width approximately 0.7 mm. The openings between struts were approximately 1.5 mm in width and 3 mm in length. The entire stent length was approximately 20 mm.

### Example 5

### Two-Layer Stent Where Each Layer Has a Different Composition

A stent was printed identically to that described in Example 1, except only two layers of polycaprolactone ink were printed. Directly on top of the polycaprolactone, two additional layers of poly(D, L-lactide) ink described in Example 4 were printed. The entire stent was cured at 55° C. for 10 minutes. The stent was easily removed in a single piece from the PTFE tube, yielding a two-layer device with a bottom, polycaprolactone layer, with a thickness of approximately 40 μm and a top, poly(D, L-lactide) layer, with a thickness of approximately 66 μm. The strut width was approximately 0.7 mm, and the openings between struts were approximately 1.5 mm in width and 3 mm in length. The entire stent length was approximately 20 mm.

### Example 6

### Stent Formed on a Water Soluble Sacrificial Substrate

Hydroxyethyl cellulose (90,000 molecular weight, Aldrich) was dissolved in deionized water at a concentration of 15% by weight. Semi-rigid Nylon 12 tubing having an outer diameter of approximately 0.4 cm (Part 1094P04 00; Legris Connectic, Inc.) was dipped into the hydroxyethyl cellulose solution and slowly withdrawn to form a coated layer. The hydroxyethyl cellulose-coated Nylon was then cured at 65° C. for 45 minutes. The resulting water soluble layer was approximately 50 μm thick.

A polycaprolactone solution was prepared as in Example 1 and applied by a Micropen™ writing device on the surface of the dried hydroxyethyl cellulose, and cured at 55° C. for 45 minutes. After curing, the entire assembly was soaked in tap water until the hydroxyethyl cellulose layer dissolved and the polycaprolactone stent structure was freed approximately 2 hours. The polycaprolactone stent was retrieved and dried under ambient conditions, yielding a cylindrical stent. The stent thickness was approximately 40 μm, the strut width was approximately 0.7 mm, and the openings between struts were approximately 1.5 mm in width and 3 mm in length. The entire stent length was approximately 20 mm.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A stent having a longitudinally-extending passage defined by a plurality of seamless strut elements with spacing between them, wherein each of the strut elements are in the form of lines defining the passage, have a thickness in the range of 30 microns to 150 microns, and are formed as at least one written layer.

2. The stent according to claim 1, wherein the strut elements form an interconnected network.

3. The stent according to claim 2, wherein the interconnected network of strut elements forms a mesh, a spiral, or a contiguous cylindrical structure.

4. The stent according to claim 1, wherein each of the strut elements are in the form of lines extending peripherally around the passage without interruption.

5. The stent according to claim 1, wherein the strut elements have a uniform thickness.

6. The stent according to claim 1, wherein the strut elements have a varying thickness.

7. The stent according to claim 1, wherein the stent has at least two written layers.

8. The stent according to claim 7, wherein the thickness of each layer is the same.

9. The stent according to claim 8, wherein the thickness of each layer is different.

10. The stent according to claim 7, wherein at least one written layer covers substantially all of the passage.

11. The stent according to claim 7, wherein at least one layer covers a portion of the passage.

12. The stent according to claim 1, wherein said at least one written layer is produced from a polymeric strut composition.

13. The stent according to claim 12, wherein said stent comprises a plurality of written layers with each different layer having the same strut composition.

14. The stent according to claim 12, wherein said stent comprises a plurality of written layers with at least two layers having different strut compositions.

15. The stent according to claim 12, wherein the strut composition comprises at least one polymer.

16. The stent according to claim 15, wherein the strut composition comprises a polymer that is biostable, bioerodable, or bioresorbable.

17. The stent according to claim 16, wherein the polymer comprises a biostable polymer selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, poly(p-xylylene), liquid crystal polymer, polymethylmethacrylate, polyhydroxyethylmethacrylate, polyphosphazene, functionalized polymers, copolymers, and blends thereof.

18. The stent according to claim 16, wherein the polymer comprises a bioerodable polymer selected from the group consisting of polyglycolide, polylactide, poly(lactide-co-glycolide), polycaprolactone, polybutylene succinate, poly(p-dioxanone), polytrimethylene carbonate, polyphosphazenes, specific polyester polyurethanes, polyether polyurethanes, polyamides, polyester amides, poly(sebacic anhydride), polyvinyl alcohol, biopolymers, gelatin, glutens, cellulose, starches, chitin, chitosan, alginates, bacterial polymers, poly(hydroxybutyrate), poly(hydroxybutyrate-co-valerate), functionalized polymers, copolymers, and blends thereof.

19. The stent according to claim 16, wherein the polymer comprises a bioresorbable polymer selected from the group consisting of polyglycolide, polylactide, poly(lactide-co-glycolide), polycaprolactone, polybutylene succinate, poly(p-dioxanone), polytrimethylene carbonate, polyphosphazenes, specific polyester polyurethanes, polyether polyurethanes, polyamides, polyester amides, poly(sebacic anhydride), polyvinyl alcohol, biopolymers, gelatin, glutens, cellulose, starches, chitin, chitosan, alginates, bacterial polymers, poly(hydroxybutyrate), poly(hydroxybutyrate-co-valerate), poly(DTE)carbonate, functionalized polymers, copolymers, and blends thereof.

20. The stent according to claim 12, wherein the strut composition further comprises a metal selected from the group consisting of magnesium, calcium, zinc, titanium, zirconium, niobium, tantalum, lithium, sodium, potassium, manganese, iron, tungsten, silicon, gold, platinum, iridium, and mixtures thereof.

21. The stent according to claim 12, wherein the strut composition further comprises a ceramic material selected from the group consisting of tricalcium phosphate, calcium potassium sodium phosphate, tricalcium phosphate, titanium oxide nitrate, hydroxyapatite, and mixtures thereof.

22. The stent according to claim 12, wherein the strut composition further comprises one or more surface active agents, rheology modifiers, lubricants, matting agents, spacers, pressure sensors, temperature sensors, chemical sensors, magnetic materials, radiopaque materials, conducting materials, therapeutic agents, or combinations thereof.

23. The stent according to claim 22, wherein the strut composition comprises a radio opaque material selected from the group consisting of magnesium, calcium, zinc, titanium, zirconium, niobium, tantalum, lithium, sodium, potassium, manganese, iron, tungsten, silicon, gold, platinum, iridium, bismuth oxychloride, bismuth bicarbonate, bismuth trioxide, barium sulfate, and mixtures thereof.

24. The stent according to claim 22, wherein the strut composition comprises a conducting material selected from the group consisting of gold, platinum, silver, nickel, copper, iron, titanium, magnesium, silicon, carbon, graphite, electrically conducting polymers, and mixtures thereof.

25. The stent according to claim 22, wherein the strut composition comprises a therapeutic agent selected from the group consisting of everolimus, sirolimus, zotarolimus, biolimus, pimecrolimus, tacrolimus, trapidil, rapamycin, paclitaxel, antithrombogenic, antiproliferative, antimotic, anti-inflammatory agents, antioxidants, anti-coagulants, anesthetics, antibiotics, and combinations thereof.

26. A method of forming a stent, the method comprising:
providing a longitudinally-extending substrate having at least an outer surface, said substrate being formed at least in part from a sacrificial material;
writing a plurality of spaced strut elements on the outer surface of the substrate, wherein the strut elements collectively form a stent with the sacrificial material being exposed at positions between the spaced strut elements, said writing being carried out with an ink composition; and
removing the sacrificial material from the substrate, leaving the stent having a longitudinally-extending passage defined by the strut elements.

27. The method according to claim 26, wherein the substrate has a tubular or cylindrical shape.

28. The method according to claim 26, wherein the strut elements have a thickness in the range of 30 microns to 150 microns.

29. The method according to claim 26, wherein each of the strut elements are in the form of lines extending peripherally around the passage without interruption.

30. The method according to claim 26, wherein said removing the sacrificial material from the substrate is carried out by melting, physically removing, disintegrating, or dissolving the sacrificial material.

31. The method according to claim 26, wherein the sacrificial material is selected from the group consisting of silicone, polytetrafluoroethylene, graphite, wax, hydroxyethyl cellulose, polyvinyl pyrrolidone, polyvinyl alcohol, polyethylene oxide, poly(ethyl oxazoline), polysaccharides, polyethylene oxide, and proteins.

32. The method according to claim 26, wherein said writing produces a stent with an interconnected network of struts.

33. The method according to claim 26, wherein said writing is carried out by screen printing, jetting, laser ablation, direct writing, pressure driven syringe delivery, inkjet or aerosol jet droplet based deposition, laser material transfer, ion-beam material transfer, tip based deposition techniques, or combinations thereof.

34. The method according to claim 33, wherein said writing is carried out by direct writing.

35. The method according to claim 33, wherein said writing is carried out with a tip based deposition technique in the form of dip pen lithography or flow based microdispensing.

36. The method according to claim 26, wherein the ink composition comprises at least one polymer.

37. The method according to claim 36, wherein the ink composition comprises a polymer that is biostable, bioerodable, or bioresorbable.

38. The method according to claim 37, wherein the polymer comprises a biostable polymer selected from the group consisting of epoxy, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, poly(p-xylylene), liquid crystal polymer, polymethylmethacrylate, polyhydroxyethylmethacrylate, polyphosphazene functionalized polymers, copolymers, and blends thereof.

39. The method according to claim 37, wherein the polymer comprises a bioerodable polymer selected from the group consisting of polyglycolide, polylactide, poly(lactide-co-glycolide), polycaprolactone, polybutylene succinate and its copolymers, poly(p-dioxanone), polytrimethylene carbonate, polyphosphazenes, specific polyester polyurethanes, polyether polyurethanes, polyamides, polyester amides, poly(sebacic anhydride), polyvinyl alcohol, biopolymers, gelatin, glutens, cellulose, starches, chitin, chitosan, alginates, bacterial polymers, poly(hydroxybutyrate), poly(hydroxybutyrate-co-valerate), functionalized polymers, copolymers, and blends thereof.

40. The method according to claim 37, wherein the polymer comprises a bioresorbable polymer selected from the group consisting of polyglycolide, polylactide, poly(lactide-co-glycolide), polycaprolactone, polybutylene succinate, poly(p-dioxanone), polytrimethylene carbonate, polyphosphazenes, specific polyester polyurethanes, polyether polyurethanes, polyamides, polyester amides, poly(sebacic anhydride), polyvinyl alcohol, biopolymers, gelatin, glutens, cellulose, starches, chitin, chitosan, alginates, bacterial polymers, poly(hydroxybutyrate), poly(hydroxybutyrate-co-valerate), poly(DTE)carbonate, functionalized polymers, copolymers, and blends thereof.

41. The method according to claim 36, wherein the ink composition further comprises a metal selected from the group consisting of magnesium, calcium, zinc, titanium, zirconium, niobium, tantalum, lithium, sodium, potassium, manganese, iron, tungsten, silicon, gold, platinum, iridium, and mixtures thereof.

42. The method according to claim 36, wherein the ink composition further comprises a ceramic material selected from the group consisting of tricalcium phosphate, calcium potassium sodium phosphate, tricalcium phosphate, titanium oxide nitrite, hydroxyapatite, and mixtures thereof.

43. The method according to claim 36, wherein the ink composition further comprises one or more surface active agents, rheology modifiers, lubricants, matting agents, spacers, pressure sensors, temperature sensors, chemical sensors, magnetic materials, radiopaque materials, conducting materials, therapeutic agents, or combinations thereof.

44. The method according to claim 43, wherein the ink composition comprises a radio opaque material selected from the group consisting of magnesium, calcium, zinc, titanium, zirconium, niobium, tantalum, lithium, sodium, potassium, manganese, iron, tungsten, silicon, gold, platinum, iridium, bismuth oxychloride, bismuth bicarbonate, bismuth trioxide, barium sulfate, and mixtures thereof.

45. The method according to claim 43, wherein the ink composition comprises a conducting material selected from the group consisting of gold, platinum, silver, nickel, copper, iron, titanium, magnesium, silicon, carbon, graphite, electrically conducting polymers, and mixtures thereof.

46. The method according to claim 43, wherein the ink composition comprises a therapeutic agent selected from the group consisting of everolimus, sirolimus, zotarolimus, biolimus, pimecrolimus, tacrolimus, trapidil, rapamycin, paclitaxel, antithrombogenic, antiproliferative, antimotic, anti-inflammatory agents, antioxidants, anti-coagulants, anesthetics, antibiotics, and combinations thereof.

47. The method according to claim 36, wherein the ink composition further comprises a solvent selected from the group consisting of paraffinic hydrocarbons, aromatic hydrocarbons, halohydrocarbons, ethers, ketones, aldehydes, esters, nitrogen-containing solvents, sulfur containing solvents, alcohols, polyhydric alcohols, phenols, water, and mixtures thereof.

48. The method according to claim 26 further comprising:
applying an overcoat layer covering at least a portion of the surface of the stent.

49. The method according to claim 48, wherein the overcoat layer comprises at least one therapeutic agent.

50. The method according to claim 49, wherein the therapeutic agent is selected from the group consisting of everolimus, sirolimus, zotarolimus, biolimus, pimecrolimus, tacrolimus, trapidil, rapamycin, paclitaxel, antithrombogenic, antiproliferative, antimotic, anti-inflammatory agents, antioxidants, anti-coagulants, anesthetics, antibiotics, and combinations thereof.

51. The method according to claim 48, wherein the overcoat layer can be selected from the group consisting of biomaterials, cellular layer, tissue layer, fabric layer, micromesh metal layer, and ink composition layer.
